(19) United States
(12) Patent Application Publication
(10) Pub. No.: US 2021/0169453 A1
(43) Pub. Date: Jun. 10, 2021

(54) THERMOACOUSTIC METHOD AND SYSTEM CONFIGURED TO INTERFACE WITH AN ULTRASOUND SYSTEM

(71) Applicant: ENDRA Life Sciences Inc., Ann Arbor, MI (US)

(72) Inventors: Christopher Bull, Saline, MI (US); Jeremy Gill, London (CA); Michael M. Thornton, London (CA)

(73) Assignee: ENDRA Life Sciences Inc., Ann Arbor, MI (US)

(21) Appl. No.: 16/705,285

(22) Filed: Dec. 6, 2019

(51) Int. Cl. A61B 8/08; A61B 8/00; G06T 7/00

(52) U.S. Cl. CPC A61B 8/5207; A61B 8/461; A61B 8/4483; A61B 5/4872; A61B 8/5246; G06T 2207/10132; G06T 2207/30056; G06T 7/0012

(57) ABSTRACT

A thermoacoustic system and method of use to receive an ultrasound system output from an ultrasound system, via an existing communication port on the ultrasound system. The thermoacoustic system includes a radio-frequency emitter, at least one thermoacoustic transducer, a processor, and a display that is integrated with the processor and configured to display an image that is a function of the ultrasound system output and data from the at least one thermoacoustic transducer. The thermoacoustic system is configured to perform an action, as a result of receiving the ultrasound system output.

### FIELD

This application relates to a method and system configured to interface with a clinical ultrasound system, and more particularly to a method and system configured to process information at a thermoacoustic imaging system that was transmitted by the clinical ultrasound system and intended for a peripheral device of the clinical ultrasound system.

### BACKGROUND

Typically, ultrasound systems that are utilized in health care have an ultrasound monitor and input devices (keyboard, touch screen, roller ball) that are integral to the system. The ultrasound monitor displays available ultrasound images derived from transducers that connect to the acquisition system of the ultrasound scanner. Any peripheral devices that are tied to the ultrasound system may have their own available displays, but these displays show images that are separate from the image on the ultrasound monitor.

A conventional approach may integrate the ultrasound image with a peripheral device on a peripheral display. A graphical element can be superimposed upon an ultrasound image and displayed on the peripheral display. The graphical element can be selected by a user in a tactile manner and used to implement a processing operation.

This conventional approach, however, assumes separate control of both the ultrasound and peripheral systems. Hence, the ultrasound and peripheral systems run in a parallel control scheme. A potentially more efficient control method would be to control ultrasound functionality from the peripheral device. Hence, there exists a need for a method and system to utilize a peripheral ultrasound device display to both control ultrasound functionality and peripheral device functionality.

### SUMMARY

A thermoacoustic system configured to receive an ultrasound system output from an ultrasound system which includes a communication port comprises: a radio-frequency emitter; at least one thermoacoustic transducer; a processor; and a display that is integrated with the processor and configured to display an image that is a function of the ultrasound system output and data from said at least one thermoacoustic transducer, wherein the thermoacoustic system is configured to perform an action as a result of receiving the ultrasound system output.

A method to utilize a thermoacoustic system that is configured to receive an ultrasound system output from an ultrasound system which includes a communication port wherein the thermoacoustic system comprises: a radio-frequency emitter; at least one thermoacoustic transducer; a processor; and a display that is integrated with the processor and configured to display an image that is a function of the ultrasound system output and data from said at least one thermoacoustic transducer, wherein the thermoacoustic system is configured to perform an action as a result of receiving the ultrasound system output comprises: utilizing the ultrasound system to acquire B-mode image data of a subject; utilizing the B-mode image to estimate a distance between a skin surface at a subcutaneous fat boundary and an intercostal muscle surface of the subject; utilizing the B-mode image to estimate a distance between the skin surface at the subcutaneous fat boundary and a liver surface of the subject; utilizing the ultrasound system to send the ultrasound system output via the communication port, wherein the ultrasound system output comprises said B-mode image data, said estimated distance between the skin surface at the subcutaneous fat boundary and a liver surface of the subject, and said estimated distance between the skin surface at the subcutaneous fat boundary and a liver surface of the subject; receiving the ultrasound system output via the communication port with the thermoacoustic system; and performing the action with the thermoacoustic system as a result of receiving the ultrasound system output.

In one embodiment, the ultrasound system output is an image file, such as a JPEG or a medical image file.

In one embodiment, the communication port is a universal serial bus (USB) port. In a separate embodiment, the communication port is a wired or wireless communication method such as TCPIP over ethernet or wirelessly networked devices over a WiFi network.

In one embodiment, the action is a thermoacoustic data acquisition which comprises the steps of: emitting pulsed radio-frequency energy with the radio-frequency emitter into a subject, wherein the subject absorbs part of the pulsed radio-frequency energy and generates thermoacoustic signals; and receiving said thermoacoustic signals with said at least one thermoacoustic transducer to generate said data.

In one embodiment, the ultrasound system output comprises a fat-layer thickness and muscle-layer thickness of the subject.

In one embodiment, the processor is configured to process said data in conjunction with the ultrasound system output to calculate a parameter.

In one embodiment, the parameter is a fat concentration of tissue of the subject.

In one embodiment, said tissue is liver tissue.

### BRIEF DESCRIPTION OF THE DRAWINGS

Embodiments will now be described more fully with reference to the accompanying drawings in which:

FIG. 1 shows a block diagram of a system with a peripheral system interfaced to an ultrasound system, according to an embodiment.

FIG. 2 shows a flow chart of a process, according to an embodiment.

FIG. 3 shows a thermoacoustic imaging system, according to an embodiment.

FIGS. 4A-4C show an ultrasound scan, according to an embodiment.

FIG. 5 shows a graphical user interface, according to an embodiment.

FIG. 6 shows a graphical user interface, according to an embodiment.

### DETAILED DESCRIPTION OF THE EMBODIMENTS

The present disclosure discusses a thermoacoustic system and method of use. The thermoacoustic system is configured to work with a pre-existing ultrasound system, without requiring any changes to the ultrasound system.

Thermoacoustic imaging describes the use of a pulsed energy source (e.g., light or radio frequency waves) to generate ultrasonic waves in tissue. The waves may be detected with conventional ultrasound equipment and used to create a high-contrast image of the tissue composition.

Photo-stimulated thermoacoustics (also referred to as photoacoustic imaging) uses visible or near-infrared light as an energy source and is well-suited for shallow-depth (e.g., 2 cm) applications, such as small-animal imaging for preclinical research, and certain shallow-depth human applications, such as breast imaging. Photoacoustic imaging does not penetrate deeply enough to render images of the human liver, kidneys, and other abdominal organs.

Radio-frequency stimulated thermoacoustics (also referred to as thermoacoustic imaging) uses radio frequency energy to penetrate deep into tissue (similar to MRI), allowing for imaging of human anatomy at depths up to about 20 cm with capabilities unavailable to traditional ultrasound and without the radiation or contrast allergy risks of CT.

A thermoacoustic system described herein transmits very short radio pulses, using a small fraction of the energy used in MRI scans, which are differentially absorbed in tissue according to water and ion (salt) content. For example, blood and organ tissues, like the liver, have a high water and ion concentration resulting in a greater signal than that from fatty tissue, with a low water and ion concentration, which result in a low signal. The radio pulses are converted by absorption in the tissue into thermoacoustic ultrasound signals, which are detected by a thermoacoustic transducer that is calibrated with a center frequency and bandwidth to maximize thermoacoustic ultrasound reception while minimizing interference. The detected thermoacoustic ultrasound is processed into measurements.

The thermoacoustic system enables the generation, display, and review of preset thermoacoustic enhanced ultrasound measurements when used with an ultrasound system for identifying gross regions of interest. The ultrasound system provides positioning (location) data, typically presented as a B-mode image on a display. The ultrasound system combines a pulsed RF source, e.g., operating at a center frequency of about 434 MHz in Europe and 915 MHz in the United States, and an RF applicator that directs the RF energy into the tissue along a desired trajectory. The emitted acoustic intensity (“response”) is detectable with a thermoacoustic transducer. The transducer and pulsed RF source (emitter) can be integrated within an imaging probe.

The imaging probe estimates the permittivity (an electrical material property) of an object (e.g., the liver, where the permittivity is strongly dependent on liver fat content). Permittivity is the measure of a material's ability to store an electric field in the polarization of the medium, expressed in Farads per meter (F/m). As lean tissue is replaced with increasing amounts of fat, its permittivity decreases.

The ultrasound system allows for output of data, including images, from a conventional ultrasound imaging system to a peripheral device, such as a printer, storage device (e.g., USB stick), or monitor. As described herein, the thermoacoustic imaging system can receive this data intended for a peripheral device of a conventional ultrasound imaging system and utilize the data for thermoacoustic imaging analysis, view, and storage.

In one embodiment, the thermoacoustic system communicates with the pre-existing ultrasound system via a pre-existing universal serial bus (USB) port on the pre-existing ultrasound system.

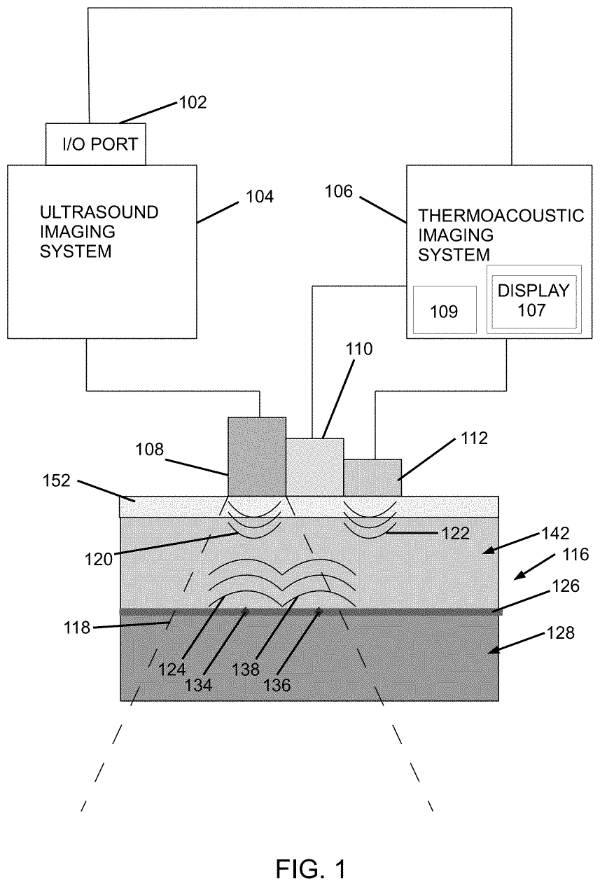

FIG. 1 shows a block diagram of a system with a peripheral system interfaced to an ultrasound system. Shown are an ultrasound input/output (I/O) port 102, ultrasound imaging system 104, thermoacoustic imaging system 106, ultrasound transducer arrays 108, B-mode image limits 118, thermoacoustic transducer 110, radiofrequency (RF) emitter 112, subject (person) 116, skin and subcutaneous fat layer 152 (both skin and subcutaneous fat shown as one layer), ultrasound waves 120, RF energy pulses 122, intercostal muscle 142, boundary 126, liver 128, boundary locations 134 and 136, and thermoacoustic multipolar signals 124 and 138.

In one embodiment, the ultrasound imaging system 104 sends a signal to ultrasound transducer arrays 108, which sends ultrasound waves 120 into subject 116. The ultrasound waves travel through the subject 116 and are reflected to give locations of skin and subcutaneous fat layer 152, intercostal muscle 142, liver 128, boundary 126 between the liver 128 and intercostal muscle 142, and boundary locations 134 and 136. The reflected sound waves are used to generate a B-mode image via the ultrasound imaging system 104 (B-mode image limits 118 shown as dashed line).

The ultrasound imaging system 104 includes an I/O port for a peripheral device. The peripheral device may be a printer, storage device (e.g., USB stick), monitor, or the like. The ultrasound imaging system 104 transmits imaging data to the peripheral device for storage, display, printing, or other function of the peripheral device. The I/O port of the ultrasound imaging system 104 may be configured as a universal serial bus (USB) port. When the peripheral device is coupled (e.g., plugged into) the I/O port, a user of the ultrasound imaging system 104 can input an instruction that causes the data to be transmitted to the peripheral device. For example, upon selecting a particular key (e.g., pressing P1 key or activating a foot pedal), data can be saved to a storage device plugged into the I/O port.

A user optionally stops imaging with the ultrasound imaging system 104, since position coordinates are now known. The thermoacoustic imaging system 106 mimics a peripheral device that is configured to communicate with the thermoacoustic imaging system 106. For example, the ultrasound imaging system 104 interacts with the thermoacoustic imaging system 106 via the I/O port as though the thermoacoustic imaging system 106 is a USB memory storage device. The ultrasound imaging system 104 and thermoacoustic imaging system 106 may use a master-slave network configuration with the ultrasound imaging system 104 functioning as master and the thermoacoustic imaging system 106 functioning as slave. The thermoacoustic imaging system 106 receives the data, which may be used for storage, display, analysis, or other function in the thermoacoustic imaging system 106.

In a separate embodiment, the thermoacoustic imaging system 106 signal mimics a USB storage device with I/O event capability and requests image file data from the ultrasound imaging system 104, then storing or otherwise utilizing the image file data as discussed in this disclosure.

The thermoacoustic imaging system 106 I/O event is configured to initiate the ultrasound imaging system 104 to (a) transfer an ultrasound image file from the ultrasound imaging system 104 to the thermoacoustic imaging system 106, (b) trigger an event on the thermoacoustic imaging system 106 (e.g., the act of saving and transferring an image from the ultrasound imaging system 104 actually causes the thermoacoustic imaging system 106 to acquire data), or (c) an I/O event on the ultrasound imaging system 104 triggers at least one processing step on the thermoacoustic imaging system 106. Examples of processing steps on the thermoacoustic imaging system 106 are emitting radio-frequency energy into a subject (person), receiving a thermoacoustic signal with a thermoacoustic transducer, calculating a subject's fat layer thickness with ultrasound data, calculating a subject's muscle layer thickness with ultrasound data, and calculating a subject's liver fat concentration with a combination of thermoacoustic data and ultrasound data.

The thermoacoustic imaging system 106 has a visual display 107 that is integrated with a processor 109 and configured to display an image that is a function of a received ultrasound signal and a received thermoacoustic transducer signal, wherein the thermoacoustic imaging system 106 is configured to receive signals from the ultrasound system 104 and receive signals from the at least one thermoacoustic transducer 110, further wherein the thermoacoustic imaging system 106 is configured to mimic one or more of the specified ultrasound system peripheral devices.

To generate thermoacoustic data, the thermoacoustic imaging system 106 initiates the RF emitter 112 to send RF energy pulses 122 into subject 116. The RF energy 122 pulses are absorbed at different rates in the skin and subcutaneous fat layer 152, intercostal muscle 142, and liver 128. The difference in RF energy absorbed between the intercostal muscle 142 and liver 128 can be measured at the boundary 126. Thermoacoustic multipolar signals 124 and 138 are generated at boundary locations 134 and 136. Thermoacoustic transducer array 110 receives the thermoacoustic multipolar signals 124 and 138 and sends the resulting data to the thermoacoustic imaging system 106, which can calculate a fat concentration in the liver 128 based upon the amplitude and optionally other characteristics of the thermoacoustic multipolar signals 124 and 138.

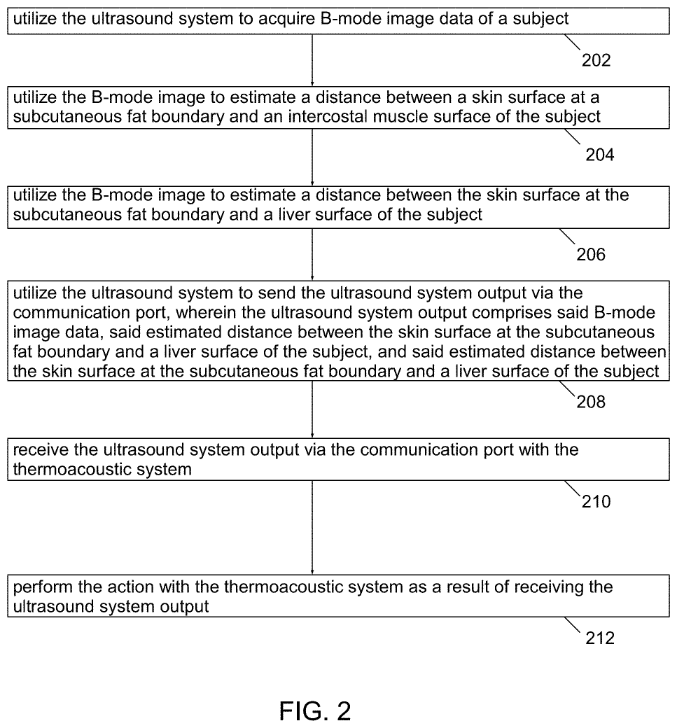

FIG. 2 shows a method embodiment. The method embodiment utilizes a thermoacoustic system configured to receive an ultrasound system output from an ultrasound system comprising a communication port, the thermoacoustic system comprising: a radio-frequency emitter; at least one thermoacoustic transducer; a processor; and a display that is integrated with the processor and configured to display an image that is a function of the ultrasound system output and data from said at least one thermoacoustic transducer, wherein the thermoacoustic system is configured to perform an action as a result of receiving the ultrasound system output.

The method embodiment in FIG. 2 shows the steps of: utilizing the ultrasound system to acquire B-mode image data of a subject (step 202); utilizing the B-mode image to estimate a distance between a skin surface at a subcutaneous fat boundary and an intercostal muscle surface of the subject (step 204); utilizing the B-mode image to estimate a distance between the skin surface at the subcutaneous fat boundary and a liver surface of the subject (step 206); utilizing the ultrasound system to send the ultrasound system output via the communication port, wherein the ultrasound system output comprises said B-mode image data, said estimated distance between the skin surface at the subcutaneous fat boundary and a liver surface of the subject, and said estimated distance between the skin surface at the subcutaneous fat boundary and a liver surface of the subject (step 208); receiving the ultrasound system output via the communication port with the thermoacoustic system (step 210); and performing the action with the thermoacoustic system as a result of receiving the ultrasound system output (step 212).

In one embodiment, a measurement obtained with the ultrasound imaging system 104 is used as an input to a processing step on the thermoacoustic imaging system 106 to calculate a parameter of interest. In one embodiment, measurements of fat and muscle thickness are used as inputs, along with thermoacoustic data acquired from the thermoacoustic transducer 204, to calculate a fat concentration in a tissue such as liver tissue.

The thermoacoustic imaging system 106 spoofs (resembles or mimics) an I/O communication method that the ultrasound imaging system 104 typically uses to communicate with a peripheral device such as a universal serial bus (USB) storage drive. In one embodiment, the ultrasound imaging system 104 functions as a master while the thermoacoustic imaging system 106 functions as a slave in a master-slave control configuration. For example, the thermoacoustic imaging system 106 will send a USB command to the ultrasound imaging system 104 which the ultrasound imaging system 104 will interpret a command to transfer data, such as a B-mode image, to the thermoacoustic imaging system 106. Handshaking can occur to verify data transfer.

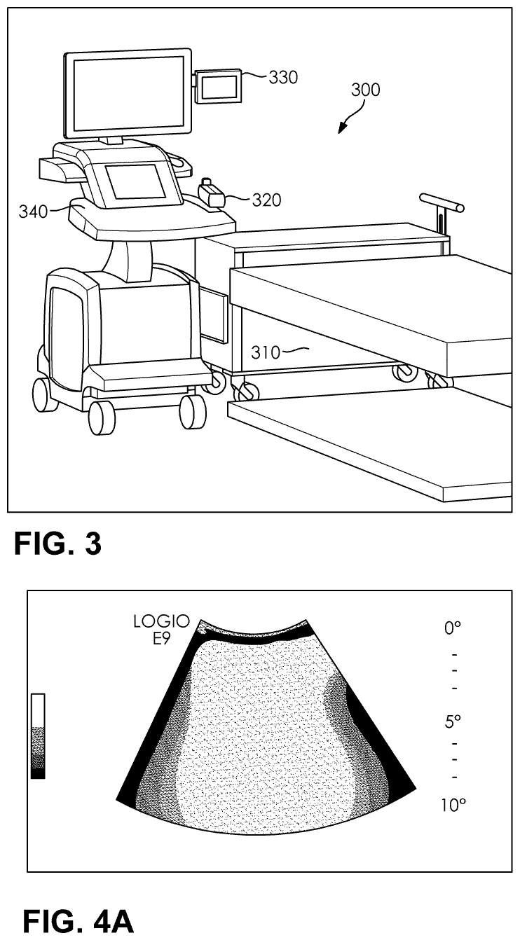

As shown in FIG. 3, the thermoacoustic imaging system 300 has three components: a console 310, a probe 320, and a monitor 330. The probe 320 comprises the RF emitter 112 and thermoacoustic transducer array 110.

The console 310 is shown as cart-mounted, but can be fixed or integrated into another component. The console 310 contains an RF source, power source, electronics, and firmware/processing.

The probe 320 is a handheld probe removable from a probe holder on an ultrasound system console 340. The handheld probe is tethered to the console 310 on a proximal end. The handheld probe has a patient-surface contacting applicator that contains the RF applicator and thermoacoustic transducer at the distal end. A set of LED lights indicate the current system status.

The monitor 300 is shown as a touchscreen monitor (may also be referred to as a “display panel”) for entering data by the user and displaying system information. The monitor 300 is integrated with the probe holder. Although the monitor is shown as a touchscreen, the monitor may be configured for use with additional or alternative inputs (e.g., stylus, mouse, keyboard).

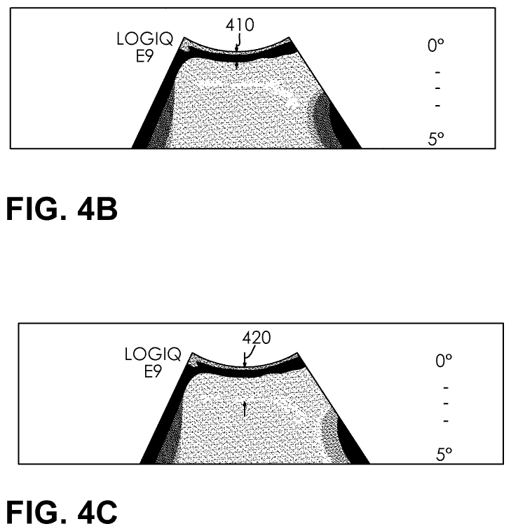

In one example, the operation of the thermoacoustic imaging system 106 interfacing with the ultrasound imaging system 104 for a subject's liver as follows. First, the ultrasound imaging system 104 acquires a B-mode image of a subject, as shown in FIG. 4A. Then, the thermoacoustic imaging system 106 sends a USB command to the ultrasound imaging system 104, which enables the ultrasound imaging system 104 to transfer B-mode image data to the thermoacoustic imaging system 106. Alternately, a user can initiate the data transfer at the ultrasound imaging system 104.

Second, the system uses the B-mode image to (a) estimate a distance between a skin surface (e.g., at subcutaneous fat boundary) of the patient and a surface of an intercostal muscle (as shown by measurement 410 from the ultrasound imaging system 104 in FIG. 4B) and (b) estimate a distance between the skin surface of the patient and the surface of a liver capsule (i.e., the subject's liver) (as shown by measurement 420 from the ultrasound imaging system 104 in FIG. 4C). Third, the thermoacoustic display (part of the thermoacoustic imaging system 106) displays (a) the estimated distance between a skin surface of the patient and a surface of an intercostal muscle and (b) the estimated distance between a skin surface of the patient and a surface of the patient's liver.

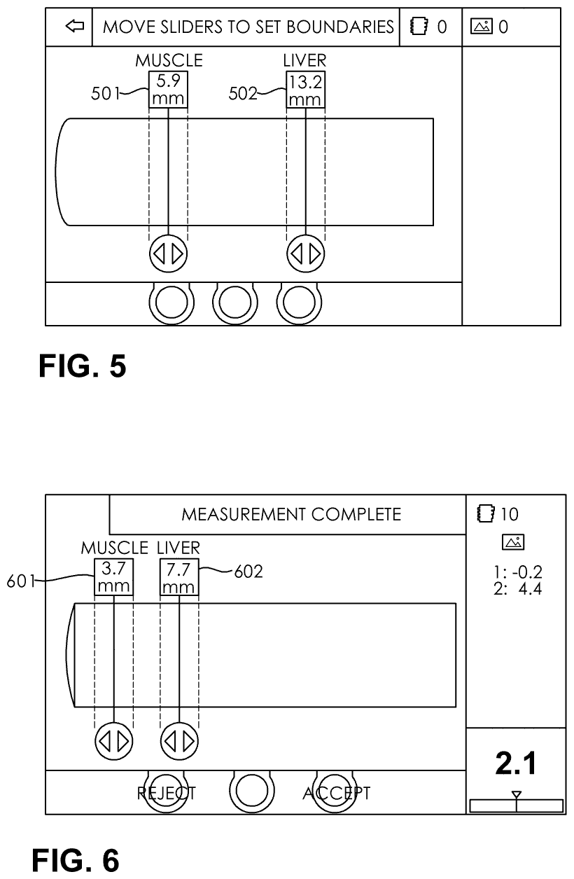

The display (which may be displayed on display 107/monitor 330 of the thermoacoustic imaging system) presents two slider bars, which a user can adjust to correspond to these distance measurements, as shown in FIG. 5 and FIG. 6.

FIG. 5 shows a skin surface to intercostal muscle distance of 5.9 mm 501 and a skin surface to liver capsule distance of 13.2 mm 502. These are initial estimates, prior to utilizing data from the B-mode image (FIG. 4A, FIG. 4B, and FIG. 4C). After utilizing B-mode image data, in FIG. 6 the sliders are set to boundaries of 3.7 mm for the a skin surface to intercostal muscle distance 601 and 7.7 mm for the skin surface to liver capsule distance 602.

Fourth, the thermoacoustic transducer 110 is positioned in a parallel orientation to the intercostal muscle.

Fifth, a switch initiates a thermoacoustic measurement with the thermoacoustic imaging system 106.

Sixth, the thermoacoustic imaging system 106 confirms that there is no interfering ultrasound, that there is sufficient contact between the thermoacoustic transducer and the patient, and that sufficient time has elapsed since the last measurement.

Seventh, the thermoacoustic imaging system 106 collects thermoacoustic data.

Eighth, the thermoacoustic imaging system 106 uses thermoacoustic data to generate calculated data such as a fat concentration in the subject's liver. As shown in FIG. 6, a graphical user interface 600 on a monitor of the thermoacoustic imaging system displays a measured thermoacoustic signal from the skin to a depth of approximately 5 cm. Two dotted lines correspond to the boundaries of the intercostal muscle and liver capsule. The graphical user interface 600 displays numerical data for the current scan and estimated permittivity (or complex relative permittivity), as well as any previous or subsequent scans from the same subject. An average value is displayed in the bottom right corner, which can be correlated to a known equivalent fat concentration of liver tissue or a proton density fat fraction (terminology used for MRI).

Ninth, a switch accepts or rejects the data. If the data is accepted, the system saves the data and allows for another scan.

Although embodiments have been described above with reference to the accompanying drawings, those of skill in the art will appreciate that variations and modifications may be made without departing from the scope thereof as defined by the appended claims.

What is claimed is:

1. A thermoacoustic system configured to receive an ultrasound system output from an ultrasound system comprising a communication port, the thermoacoustic system comprising:
a radio-frequency emitter;
at least one thermoacoustic transducer;
a processor; and
a display that is integrated with the processor and configured to display an image that is a function of the ultrasound system output and data from said at least one thermoacoustic transducer, wherein the thermoacoustic system is configured to perform an action as a result of receiving the ultrasound system output.

2. The system of claim 1, wherein the ultrasound system output is an image file.

3. The system of claim 1, wherein the communication port is a universal serial bus port.

4. The system of claim 1, wherein the action is a thermoacoustic data acquisition which comprises the steps of:
emitting pulsed radio-frequency energy with the radio-frequency emitter into a subject, wherein the subject absorbs part of the pulsed radio-frequency energy and generates thermoacoustic signals; and
receiving said thermoacoustic signals with said at least one thermoacoustic transducer to generate said data.

5. The system of claim 4, wherein the ultrasound system output comprises a fat-layer thickness and muscle-layer thickness of the subject.

6. The system of claim 5, wherein the processor is configured to process said data in conjunction with the ultrasound system output to calculate a parameter.

7. The system of claim 6, wherein the parameter is a fat concentration of tissue of the subject.

8. The system of claim 7, wherein said tissue is liver tissue.

9. A method to utilize the system of claim 1, the method comprising:
utilizing the ultrasound system to acquire B-mode image data of a subject;
utilizing the B-mode image to estimate a distance between a skin surface at a subcutaneous fat boundary and an intercostal muscle surface of the subject;
utilizing the B-mode image to estimate a distance between the skin surface at the subcutaneous fat boundary and a liver surface of the subject;
utilizing the ultrasound system to send the ultrasound system output via the communication port, wherein the ultrasound system output comprises said B-mode image data, said estimated distance between the skin surface at the subcutaneous fat boundary and a liver surface of the subject, and said estimated distance between the skin surface at the subcutaneous fat boundary and a liver surface of the subject;
receiving the ultrasound system output via the communication port with the thermoacoustic system; and
performing the action with the thermoacoustic system as a result of receiving the ultrasound system output.

10. The method of claim 9, wherein the ultrasound system output is an image file.

11. The method of claim 9, wherein the communication port is a universal serial bus port.

12. The method of claim 9, wherein the action is a thermoacoustic data acquisition which comprises the steps of:
emitting pulsed radio-frequency energy with the radio-frequency emitter into a subject, wherein the subject absorbs part of the pulsed radio-frequency energy and generates thermoacoustic signals;
receiving said thermoacoustic signals with said at least one thermoacoustic transducer to generate said data.

13. The method of claim 12, wherein the ultrasound system output comprises a fat-layer thickness and muscle-layer thickness of the subject.

14. The method of claim 13, wherein the processor is configured to process said data in conjunction with the ultrasound system output to calculate a parameter.

15. The method of claim 14, wherein the parameter is a fat concentration of tissue of the subject.

16. The method of claim 15, wherein said tissue is liver tissue.
